(19) United States
(12) Patent Application Publication
(10) Pub. No.: US 2022/0339409 A1
(43) Pub. Date: Oct. 27, 2022

(54) DEVICE AND METHOD FOR SHAPING A WIRE

(71) Applicant: LES SOLUTIONS MÉDICALES SOUNDBITE INC., Saint-Laurent (CA)

(72) Inventors: Maude Laforest, Québec (CA); Gholamreza Mirshekari, Montréal, (CA); Louis-Philippe Riel, Magog (CA); Gautier Halbin, Québec (CA)

(21) Appl. No.: 17/765,314

(22) PCT Filed: Oct. 16, 2020
(86) PCT No.: PCT/IB2020/059776

§ 371 Date: Mar. 30, 2022

Related U.S. Application Data
(60) Provisional application No. 62/916,315, filed on Oct. 17, 2019.

(51) Int. Cl. A61M 25/09

(52) U.S. Cl. CPC A61M 25/09; A61M 2025/09108; A61M 2207/10

(57) ABSTRACT

A device and a method for shaping a wire used in medical procedures, such as a guidewire used in conjunction with catheters. The device includes a body having a working face, and the body includes a wire-shaping portion, the wire-shaping portion having an aperture defined therein which extends from the working face along an aperture axis. The apertures include a flared section and a tip receiving section connected to the flared section, where the tip receiving section is shaped and sized for receiving a tip of the wire therein and maintaining a position of the tip of the wire while a bending force is exerted on the wire towards the working face. In one embodiment, the wire-shaping portion has a mound-shaped portion projecting from the working face from which the aperture extends, which enables to curve the wire in a smooth shape that follows the mound-shaped portion.

### TECHNICAL FIELD

The present technology relates to the field of wires used in the medical field, and more particularly to a device and a method for shaping wires such as a guidewire that could be used in conjunction with catheters.

### BACKGROUND

Percutaneous medical intervention is a minimally invasive procedure that relies mostly on the use of catheters as well as on different types of guidewires. In at least some surgical procedures, a tip of the guidewire may need to be shaped, bent or curved in order to improve, amongst other things, the steerability of the guidewire as it progresses along the vascular system, thereby reducing the risk of injuring the blood vessels.

Shaping tips of guidewires require in some instances a medical practitioner to manually shape the guidewires. Considering that some guidewires are fine and may be difficult to grip for shaping, especially when coated with lubricious material to make them pass more easily through the catheter and blood vessels, it may be challenging for a medical practitioner to manually shape the guidewires in a desired form. In some instances, guidewires may be damaged during the manual bending or curving process or damaged by other tools used by practitioners that may not be necessarily suitable for use with guidewires.

Therefore, there is a need for a device and method for shaping a wire such as a guidewire.

### SUMMARY

Embodiments of the present technology has been developed based on the inventors' appreciation that there is a need for a device that is adapted to and convenient for shaping a wire used during a medical procedure, such as a guidewire that could be used in conjunction with catheters.

Thus, embodiments of the present technology are directed to a device for and a method of shaping a wire.

In accordance with a broad aspect of the present technology, there is provided a device for shaping a wire comprising: a body having a working face, the body comprising a wire-shaping portion, the wire-shaping portion having an aperture defined therein, the aperture extending from the working face along an aperture axis, the aperture comprising: a flared section, and a tip receiving section connected to the flared section, the tip receiving section being shaped and sized for receiving a tip of the wire therein and maintaining a position of the tip of the wire while a bending force is exerted on the wire.

In one embodiment of the device, the wire-shaping portion further comprises a mound-shaped portion projecting from the working face, and the aperture extends from the mound-shaped portion.

In one embodiment of the device, the mound-shaped portion has a conical frustum shape, the conical frustum having: a lower base and an upper base, the lower base being located below the upper base.

In one embodiment of the device, the mound-shaped portion has a pyramidal-frustum shape.

In one embodiment of the device, the mound-shaped portion has an arcuate top portion.

In one embodiment of the device, the mound-shaped portion has a helicoid-shaped surface.

In one embodiment of the device, a height of the mound-shaped portion varies along a circumference thereof.

In one embodiment of the device, the mound-shaped portion is a plurality of mound-shaped portions.

In one embodiment of the device, the flared section has a conical frustum shape, the conical frustum having: a lower base, and an upper base, the upper base being located below the lower base.

In one embodiment of the device, the axis of the aperture is orthogonal to the working face.

In one embodiment of the device, the flared section is a plurality of flared sections each extending from a respective one of the plurality of mound-shaped portions.

In one embodiment of the device, the aperture extends along only a section of a thickness of the body, the tip receiving section extending up to an abutment wall for abutting the tip of the wire thereon.

In one embodiment of the device, the abutment wall is flat.

In one embodiment of the device, the tip receiving portion has a cylindrical shape.

In one embodiment of the device, the device further comprises a plurality of visual indicators each for identifying respective dimensions of the aperture and the tip-receiving section.

In one embodiment of the device, the wire is a guidewire for guiding a catheter.

In one embodiment of the device, the body is made at least partially of one of: a medical-grade material, a biocompatible material and a sterilizable material.

In one embodiment of the device, the material is polyether ether ketone.

In accordance with another broad aspect of the present technology, there is provided a method for shaping a wire. The method comprises: providing a device comprising a body having a working face and a wire-shaping portion, the wire-shaping portion having an aperture defined therein, the aperture extending from the working face towards the bottom face along an axis, the aperture comprising: a flared section, and a tip receiving section connected to the flared section, the tip receiving section being shaped and sized for receiving a tip of the wire therein and maintaining a position of the tip of the wire while a bending force is exerted on the wire. The method comprises inserting the wire into the aperture such that a portion of the wire is positioned within the tip receiving section of the aperture. The method comprises exerting a force on a working section of the wire towards the working face, the working section of the wire being located outside of the aperture, thereby shaping the wire.

In one embodiment of the method, the inserting the wire into the aperture comprises positioning a distal end of the wire into the tip receiving section of the aperture

In one embodiment of the method, the inserting the wire into the aperture comprises abutting the distal end of the wire against an end wall of the tip receiving section of the aperture.

In one embodiment of the method, the inserting the wire into the aperture comprises positioning a distal end of the wire through the body.

In one embodiment of the method, the exerting the force comprises abutting at least a portion of the working section of the wire against the working face of the body.

In one embodiment of the method, the wire-shaping portion further comprises a mound-shaped portion, and the exerting the force towards the working face comprises abutting another portion of the working section of the wire on the mound-shaped portion.

In one embodiment of the method, the wire is a guidewire for guiding a catheter.

While the present specification refers to a device for shaping a guidewire, it should be understood that the device could be adapted for shaping any wire-shaped objects used during medical procedures, such a mechanical waveguide for example.

### BRIEF DESCRIPTION OF THE DRAWINGS

Further features and advantages of the present technology will become apparent from the following detailed description, taken in combination with the appended drawings, in which:

FIG. 1 is a top plan view of a device for shaping a wire, in accordance with a first non-limiting embodiment.

FIG. 2 is a right-side elevation view of the device illustrated in FIG. 1.

FIG. 3 is cross sectional view of the device illustrated in FIG. 1, taken along a plane A-A.

FIG. 4 is a zoomed cross-sectional view of an aperture of the device illustrated in FIG. 3.

FIG. 5 is a cross-sectional view of a device for shaping a wire, in accordance with a second non-limiting embodiment.

FIG. 6 is a perspective view taken from the top left of a device for shaping a wire, in accordance with a third non-limiting embodiment.

FIG. 7 is a perspective view taken from the top front right side of the device of FIG. 6.

FIG. 8 is a top plan view of the device of FIG. 6.

FIG. 9 is a perspective view taken from the top left side of a first and a second aperture of the device of FIG. 6.

FIG. 10 is a cross-sectional view taken of the first aperture of the device of FIG. 6 taken along the plane B-B.

FIG. 11 is a perspective view taken from the top left side of a second aperture of the device of FIG. 6.

FIG. 12 is a cross-sectional view of the second aperture of the device of FIG. 6 taken along the plane B-B.

FIG. 13 is a perspective view taken from the top left of the third and fourth apertures of the device of FIG. 7.

FIG. 14 is a cross-sectional view of the third aperture of the device of FIG. 7 taken along the plane B-B.

FIG. 15 is a perspective view taken from the top left of fourth aperture of the device of FIG. 7.

FIG. 16 is a cross-sectional view of a fourth aperture of the device of FIG. 7 taken along the plane B-B.

FIG. 17 is a flow chart illustrating a method for shaping a wire, in accordance with one embodiment.

It will be noted that throughout the appended drawings, like features are identified by like reference numerals.

### DETAILED DESCRIPTION

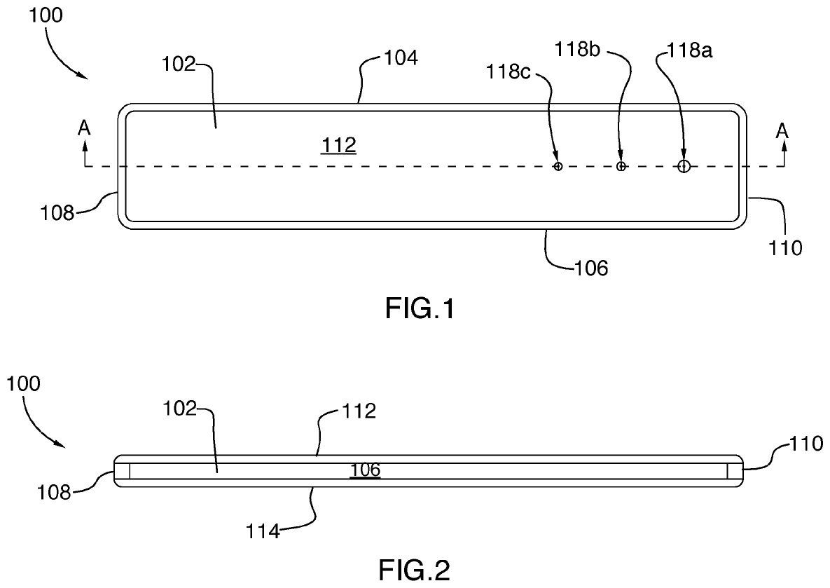

FIG. 1 to FIG. 4 illustrate a first non-limiting embodiment of a device 100 for shaping a given section of a wire such as a guidewire.

The device 100 may be used for shaping a tip section of a guidewire for example, i.e. for modifying the shape of the tip section of a guidewire. In another example, the device 100 may be used for shaping a central section of a guidewire.

The device 100 comprises an elongated body 102 having a substantially rectangular shape (e.g. a rectangular parallelepiped). The body 102 extends laterally between a first lateral face 104 and a second lateral face 106, and extends longitudinally between a first longitudinal face 108 and a second longitudinal face 110. The body 102 extends transversally between a first transverse face 112 or top face 112 (referred hereinafter as the working face 112) and a second transverse face 114 or bottom face 114 opposite to the working face 112. The distance between the working face 112 and the bottom face 114 defines the thickness 116 of the body 102.

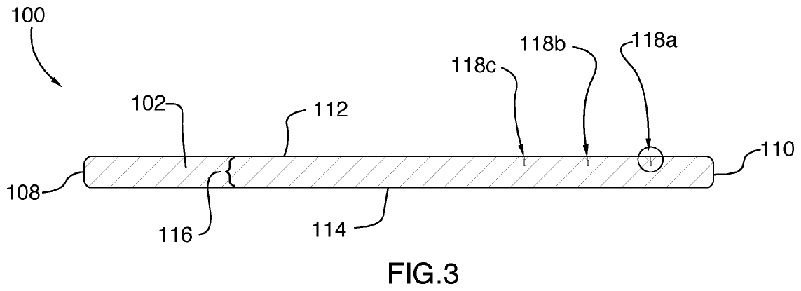

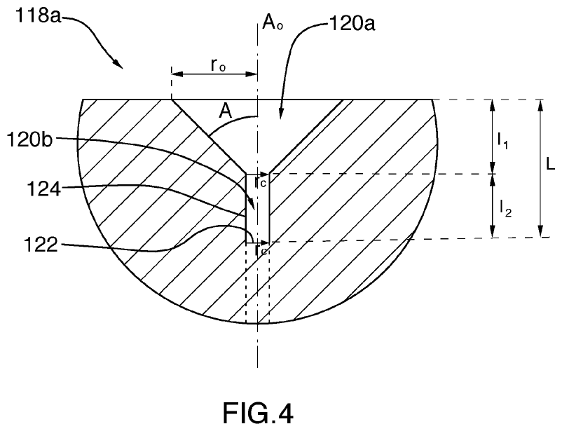

As best seen in FIG. 1 and FIG. 3, the body 102 comprises three wires-shaping portions 118, i.e. a first aperture 118a, a second aperture 118b and third aperture 118c, defined therein. Each of the first aperture 118a, the second aperture 118b and the third apertures 118c are defined in the body 102 and extends from the working face 112 towards the bottom face 114 along a respective axis Aₒ (as depicted in FIG. 4) through a portion of the thickness of the body 102.

In the illustrated embodiment, the three apertures 118a, 118b and 118c are positioned along a longitudinal axis AA located at equal distance from the first lateral face 104 and the second lateral face 106. However, it is contemplated that other locations for each of the three apertures 118a, 118b and 118c are possible. In one embodiment, the apertures 118a, 118b and 118c may not all be aligned along the same longitudinal axis. As a non-limiting example, the first aperture 118a may be located adjacent to the first lateral face 104, the second aperture 118b may be located at equal distance from the first lateral face 104 and the second lateral face 106, and the third aperture 118c may be located adjacent to the second lateral face 106. Similarly, while the apertures 118a, 118b and 118c are depicted as being defined on the same face of the body 102, i.e. on the working face 112, it should be understood that the apertures 118a, 118b and 118c may be defined on different faces of the body 102, such as on the first lateral face 104, the second lateral face 106 and/or the bottom face 114.

In one embodiment such as the embodiment illustrated in FIG. 1, each of the first, the second and the third apertures 118a, 118b and 118c have a different respective radius so as to accommodate and shape wires having different cross-sectional sizes such as cylindrical wires having different radiuses. It should be understood that while the apertures are depicted as having a circular cross-section, they could have any adequate shape adapted to receive a guidewire therein.

As best seen in FIGS. 3 and 4, each of the apertures 118a, 118b and 118c is funnel-shaped and extends from the working face 112 towards the bottom face 114 along the axis Aₒ (as depicted in FIG. 4).

The apertures 118a, 118b and 118c being equivalent to one another, only the aperture 118a will be described. It will be appreciated that a similar description also applies to the apertures 118b and 118c with proper adaptation, notably as to the dimensions such as the radiuses, the length and chamfer angle A of the apertures.

As illustrated in FIG. 4, the aperture 118a extends along the axis A₀ and comprises a flared or conical frustum shaped section 120a, i.e. shaped as a truncated cone, extending from the working face 112, and a tip receiving or tubular section 120b extending from the flared section 120a towards the bottom face 114.

The radius rₒ of the flared section 120a decreases from the working face 112 towards the bottom face 114 until being equal to the radius r_{c} of the tubular section 120b, while the radius r_{c} of the tubular section 120b remains constant along the length thereof. The length of the aperture 118a along the axis A₀ is referred to as L and is equal to the summation of the length l₁ of the flared section 120a and the length l₂ of the tubular section 120b.

As illustrated in FIG. 4, the geometrical characteristics of the aperture 118a are defined by the length l₁ of the flared section 120a, the length l₂ of the tubular section 120b, a chamfer angle A, a radius rₒ of the flared section 120a and/or a radius r_{c} of the tubular section 120b, which indirectly also dictate the characteristics of the guidewires that may be shaped, bent or curved using the aperture 118a of the device 100. As a result, any variation in the values of the geometrical parameters of the aperture 118a (e.g. A, l₁, l₂, rₒ and/or r_{c}) changes the resulting configuration for the shaped guidewire. Therefore, a desired shape for the tip section of the guidewire may be obtained by adequately adjusting the geometrical characteristics or parameters of the aperture 118a, as understood by the person skilled in the art.

In use and as described in greater detail hereinafter, the tip section of a guidewire to be shaped is inserted into an adequate one of the apertures 118a, 118b and 118c. The adequate aperture 118a, 118b, 118c is chosen as a function of the characteristics of the tip section of the guidewire to be shaped and the characteristics of the apertures 118a, 118b and 118c such as the radius rₒ of the flared section 120a, the radius r_{c} of the tubular section 120b and/or the chamfer angle A. Once the tip section of the guidewire is inserted into the tubular section 120b, the guidewire is bent so that a portion of the guidewire extending outside from the aperture 118a, 118b, 118c (referred hereinafter as the working section of the guidewire) comes in physical contact with the working face 112. In one embodiment, the guidewire is inserted into the tubular section 120b such that its tip section abuts the end wall 122 of the tubular section 120b.

In one embodiment, the chamfer angle A is chosen so as to facilitate the insertion of the tip of a guidewire into the aperture 118a, 118b, 118c by guiding it towards the tubular section 120b. In the same or another embodiment, the chamfer angle A is chosen so as to enable shaping, bending, or curving the guidewire without damaging it by providing a clearance so that the guidewire does not contact the body 102 in the region of the guidewire being shaped, bent, or curved.

While the device 100 comprises three apertures 118a, 118b and 118c, it should be understood that the number of apertures may vary (i.e. it may be a plurality of apertures) as long as the device 100 comprises at least one aperture. It should also be understood that the location of the aperture(s) on the body 102 and/or the shape and characteristics of the aperture(s) may also vary.

While the body 102 has a rectangular cross-sectional shape in the illustrated embodiment, it should be understood that the cross-section of the body 102 may be provided with any other adequate cross-sectional shape such as a square or circular cross-sectional shape. The body 102 may also be provided with an ergonomic shape sized and shaped for fitting an average user's hand. In one embodiment, the working face 112 may be convex or concave around the region surrounding the apertures 118a, 118b and 118c. In one embodiment, the working face 112 may be conical, i.e., it may have a constant slope towards or away from the transverse face 114. As a non-limiting example, the working face 112 may be a curved section around the region surrounding an aperture, as seen from a side elevation view. Additionally, any one of the apertures 118a, 118b and 118 may be at the center of the curved region of the working face 112 or it may be offset from its center. In one embodiment, a region of the working face 112 may not be symmetrical around the axis of the aperture 118.

In an alternative embodiment, the bottom face 214 of the body 102 may also have a curved surface.

It should further be understood that the geometrical characteristics of each of the apertures 118a, 118b and 118c may vary so as to accommodate the size and shape of any given respective wire to be shaped

In one embodiment, each of the apertures 118a, 118b and 118c is provided with a chamfer angle A comprised between about 30 and about 60 degrees, an aperture length L comprised between about 0.039 and about 0.118 inch, a radius rₒ comprised between about 0.0315 and about 0.059 inch, and a radius r_{c} comprised between about 0.005 and about 0.010 inch. It should be noted that the length L and/or the radius rₒ and/or the radius r_{c} may increase for wires with larger dimensions and/or different intended use.

In one embodiment, each aperture 118a, 118b and 118c has a chamfer angle A of about 45 degrees, an aperture length L of about 0.079 inch and a radius r_{c} of about 0.0065 inch

In one embodiment, the chamfer angle A is chosen as a function of a desired angle for a curvature of the guidewire, the length L is chosen as a function of the desired length of the guidewire to be curved, and/or the radius r_{c} is chosen as a function of the tip diameter of the guidewire to be curved.

While the aperture 118a, 118b and 118c is provided with a shape, it should be understood that the aperture 118a, 118b and 118c may be provided with any other adequate shape. As a non-limiting example, a respective one of the apertures 118a, 118b and 118c may be provided with: a conical shape, a parabolic shape, a pyramidal shape, or the like, i.e. each aperture 118a, 118b and 118c may have a different shape.

While the apertures 118a, 118b and 118c each extend through a section of the thickness 116 of the body 102, it should be understood that the apertures 118a, 118b and/or 118c may extend entirely through the whole thickness 116 of the body 102.

While the apertures 118a, 118b and 118c are provided with the same shape, i.e. a countersunk shape, it should be understood that the at least two of the apertures 118a, 118b and 118c may be provided with different shapes.

While the apertures 118a, 118b and 118c each extend along an axis Aₒ that is orthogonal to the working face 112, it should be understood that at least one of the apertures 118a, 118b and 118c may extend along an axis that is not orthogonal to the working face 112.

In an embodiment in which the aperture 118a, 118b, 118c does not extend through the entire thickness of the body 102, the end wall 122 may be flat as illustrated in FIG. 4. However, it should be understood that the end wall 122 may have any other adequate shape such as a rounded shape.

In one embodiment, the wall defining the aperture 118a, 118b, 118c is provided with a wire fitting curved groove so that when force is applied by the user into the guidewire fitting groove, the guidewire is aligned, and multiple bends may be co-directional.

While the aperture 118a, 118b, 118c is symmetrical around a central axis, it should be understood that the aperture 118a, 118b, 118c may be asymmetrical. For example, the aperture 118a, 118b, 118c may have different angles in different directions such as the user could create various shapes using a same aperture depending on the direction in which the guidewire is bent.

In one embodiment, the body 102 may further be provided with a side port hole that may be used for reaching the bottom of the aperture 118a, 118b, 118c to facilitate cleaning and/or removal of particles after machining of the body 102.

It should be understood that any adequate method for fabricating the body 102 may be used. For example, etching, 3D printing, machining, molding or engraving may be used for fabricating the body 102.

In one embodiment, the body 102 is a one-piece construction etched, 3D printed, machined, molded or engraved.

In one embodiment, the body 102 is at least partially made from a material that is biocompatible, sterilizable and/or of medical grade. In one embodiment, the whole body 102 is made of a material that is biocompatible, sterilizable and/or of medical grade. In another embodiment, only a portion or only portions of the body 102 is (are) made of a material that is biocompatible, sterilizable and/or of medical grade. For example, the portion of the body 102 that comprises the aperture(s) made be made of a material that is biocompatible, sterilizable and/or of medical grade while the remaining of the body 102 may be made of any other adequate material which would not damage a guidewire in contact with the body 102 and create any residue, debris or particles from the guidewire or the body 102.

In one embodiment, the body 102 is made from glass or metal.

In one embodiment, the body 102 is made from a polymer such as polyether ether ketone.

In one embodiment, the body 102 comprises one or more visual indicators (not shown) for facilitating the identification and/or selection of the apertures of the plurality of apertures. For example, a visual indication of the chamfer angle A and/or radius rₑ of the tubular section 120b for each aperture 118a, 118b, 118c may be provided on the working face 112 adjacent to the respective aperture 118a, 118b, 118c. As a non-limiting example, the visual indicators may be added to the body 102 by using laser welding, engraving, or may be in the form of stickers disposed on the body 102.

In one embodiment, the device 100 includes one or more features to improve handling, ergonomics, and grip (e.g. anti-slip grip). In a first non-limiting example, the device 100 may further comprise a handle projecting from the lateral face 104 or 106 or from the longitudinal face 108 or 110. In a second non-limiting example, the device 100 may further comprise a stippling pattern to enhance gripping of the device 100. In a third non-limiting example, the device 100 may further comprise a gripping material such as tape. The device 100 may comprise different visual colors or patterns to differentiate the different device features or regions to guide the user.

In one embodiment, the length of the lateral faces 104 and 106 is about 5.00 inches, the length of the longitudinal faces 108 and 110 is about 0.50 inch, and the distance between the working face 112 and the bottom face 114 (i.e. the thickness 116 of the body 102) is about 0.25 inch. It will be appreciated, however, that the device 100 may have any other size and shape as long as it enables a user to grip and handle the device 100 for shaping, bending, or curving a guidewire.

While the length L of the aperture 118a, 118b, 118c is less than the thickness 116 of the body 102, it should be understood that the length L of at least one of the apertures 118a, 118b and 118c may be equal to the thickness 116 of the body 102 so as to extend through the body 102. In such a configuration, a guidewire may pass from the lateral face 104 to the lateral face 106 through the thickness 116 of the body 102.

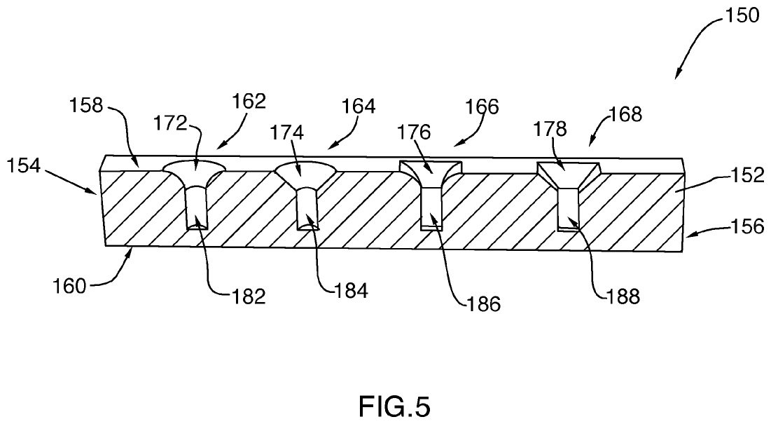

With reference to FIG. 5, there is illustrated a second non-limiting embodiment of a device 150 for shaping a section of a guidewire.

The device 150 comprises an elongated body 152 having a rectangular cross-sectional shape. The body 152 extends longitudinally between a first longitudinal face 154 and a second longitudinal face 156. The body 152 further extends transversally between a first transverse face 158 or top face 158 (referred hereinafter as the working face 158) and a second transverse face 160 or bottom face 160 opposite to the working face 158. The distance between the working face 158 and the transverse face 160 defines the thickness of the body 152.

The body 152 is provided with four apertures 162, 164, 166 and 168 which each extend from the working face 158 towards the transverse face 160 through a portion of the thickness of the body 152. The apertures 162, 164, 166 and 168 are each provided with a different respective shape for providing guidewires with different curvatures.

The apertures 162, 164, 166 and 168 are each provided with a flared portion 172, 174, 176, 178, respectively, which extends from the working face 158, and a tip receiving portion 182, 184, 186, 188, respectively, which extends from the respective flared portion 172, 174, 176, 178 further towards the transverse face 160.

The flared portion 172 of the aperture 162 has a conical frustum shape and has a concave wall. The flared portion 174 of the aperture 164 has a conical frustum shape with a straight wall. The flared portion 176 of the aperture 166 has a pyramidal frustum shape, i.e. a truncated pyramid, with concave walls. The flared portion 178 of the aperture 168 has a pyramidal frustum shape provided with straight walls.

The tip receiving portions 182 and 184 of the apertures 162 and 164 are each provided with a cylindrical or tubular shape while the tip receiving portions 186 and 188 of the apertures 166 and 168 are each provided with a rectangular shape.

While the apertures 162, 164, 166 and 168 are provided with the same length along the transverse axis, it should be understood that at least two of the apertures 162, 164, 166 and 168 may have a different length.

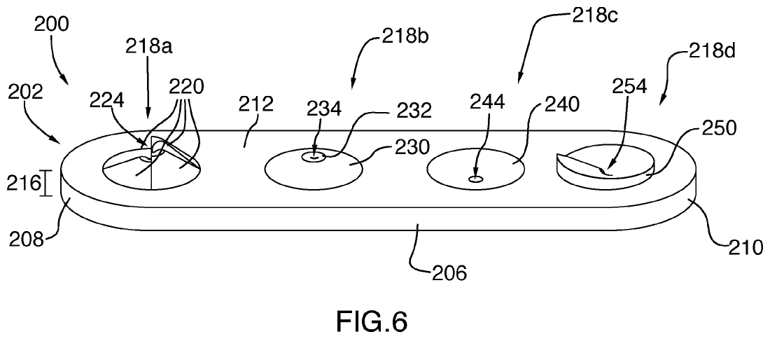

It should also be understood that the shapes of the apertures 162, 164, 166 and 168 illustrated in FIG. 6 are exemplary only and that any adequate shape for an aperture designed to provide a desired curvature for a guidewire could be used.

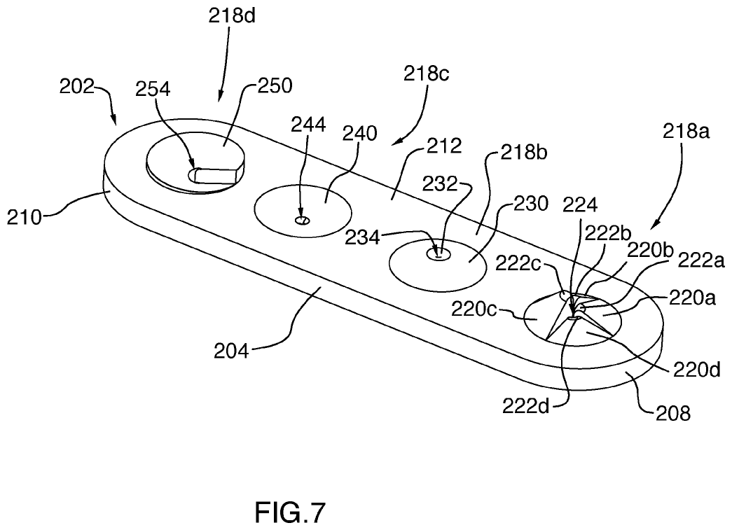

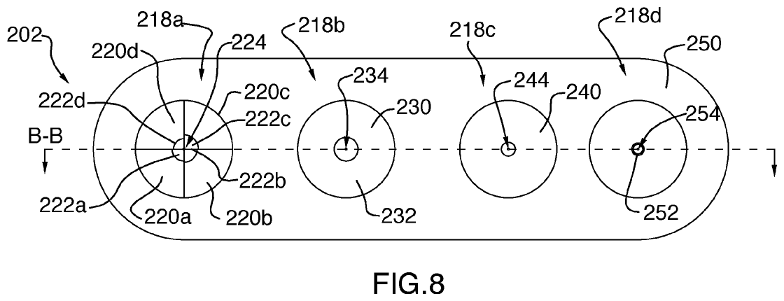

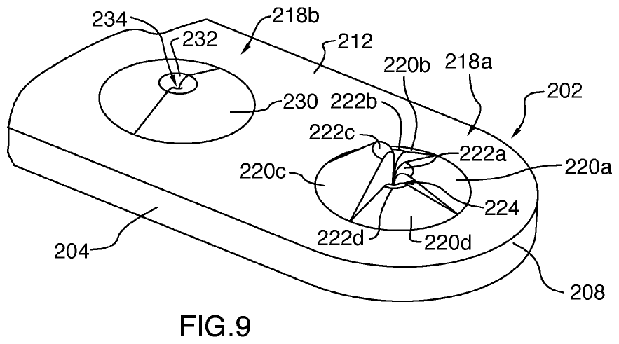

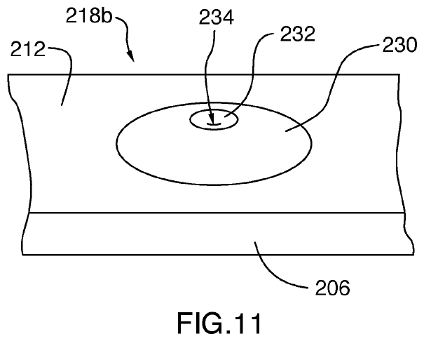

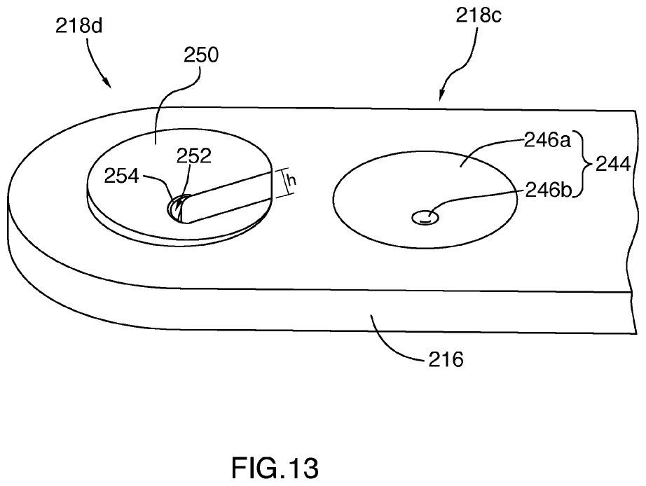

Now turning to FIGS. 6 to 16, there is depicted a third non-limiting embodiment of a device 200 for shaping guidewires.

The device 200 comprises an elongated body 202 having a rounded rectangular shape, i.e., a rectangular shape provided with rounded longitudinal ends. The body 202 extends laterally between a first lateral face 204 and a second lateral face 206. The body 202 extends longitudinally between a first longitudinal face 208 and a second longitudinal face 210. The body 202 further extends transversally between a first transverse face 212 or top face 212 (referred hereinafter as the working face 112) and a second transverse face 214 or bottom face 214 opposite to the working face 212. The distance between the working face 212 and the transverse face 214 defines the thickness 216 of the body 202.

The device 200 comprises four wire-shaping portions 218: a first wire-shaping portion 218a, a second wire-shaping portion 218b, a third wire-shaping portion 218c, and a fourth wire-shaping portion 218d.

The first wire-shaping portion 218a comprises a first set of mound-shaped portions 220 and has a first aperture 224 defined therein.

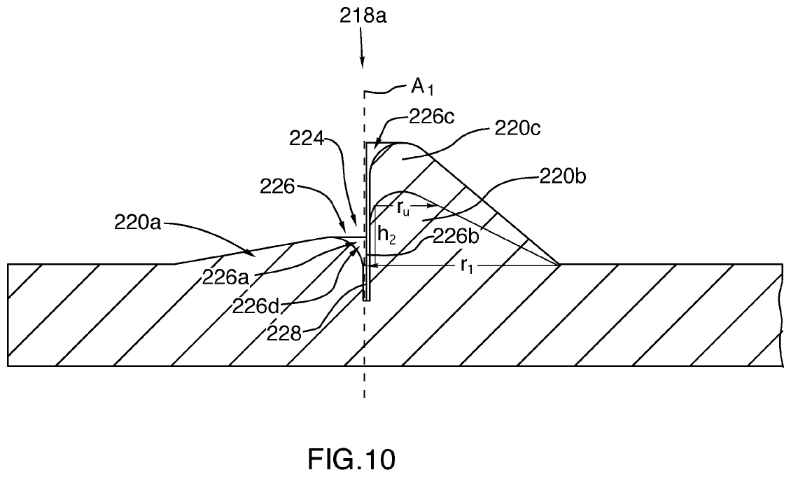

The first set of mound-shaped portions 220 project from the working face 212 of the body 202 away from the bottom face 214 along a first axis A₁ (only depicted in FIG. 10).

As best seen in FIGS. 6 to 9, the first set of mound-shaped portions 220 comprises four mound-shaped portions 220a, 220b, 220c, 220d, each being shaped as a portion of a conical frustum defined by two transversal planes perpendicular to the working face 212 (not depicted), i.e. a transversal portion of a truncated cone, each having a respective lower base radius r_{b}, a respective upper base radius rᵤ, and a respective height h₂, as illustrated in FIG. 10. In the illustrated embodiment, each of the first set of mound-shaped portions 220a, 220b, 220c, 220d has a different height h₂ and different upper base radius rᵤ but has the same lower-base radius r_{b}. It is contemplated that in an alternative embodiment, at least one of the first set of mound-shaped portions 220a, 220b, 220c, 220d may have a different lower base radius r_{b}. While the first set of mound-shaped portions 220 is depicted as having four mound-shaped portions 220a, 220b, 220c, 220d, it should be understood that the first set of mound-shaped portions 220 may have less than four mound-shaped portions or more than four mound-shaped portions without departing from the scope of the present technology. Each mound-shaped portion of the first set of mound-shaped portions 220 may generate a different tip curve based on the respective lower base radius r_{b}, the respective upper base radius rᵤ, and the respective height h₂,

Each mound-shaped portion 220a, 220b, 220c, 220d has a respective top portion 222a, 222b, 222c, 222d. The top portions 222a, 222b, 222c and 222d are smooth and generally have an arcuate shape. The shape of each top portion 222a, 222b, 222c, 222d allows giving a smooth shape to the working section of the guidewire that abuts on the respective top portion 222a, 222b, 222c, 222d when a force is exerted on the guidewire towards the working face 212 when the tip of the guidewire is inserted into the first aperture 224.

The first aperture 224 extends from the first set of mound-shaped portions 220 above the working face 212 towards the transverse face 214.

As best seen in FIG. 10, the first aperture 224 the respective top portions 222a, 222b, 222c and 222d of the mound-shaped portions 220a, 220b, 220c and 220d.

The first aperture 224 has a flared section 226 and a tip receiving section 228.

The flared section 226 includes a first flared section 226a, a second flared section 226b, a third flared section 226c, and a fourth flared section 226d (only two depicted in FIG. 10). The flared sections 226a, 226b, 226c and 226d project circumferentially from the tip receiving section 228.

Each flared section 226a, 226b, 226c, 226d extends from its respective top portion 222a, 222b, 222c, 222d towards the bottom face 214.

Each flared section 226a, 226b, 226c, 226d is shaped as a portion of a respective conical frustum, i.e. a portion of a cone truncated by two transversal planes perpendicular to the working face 212 (not depicted) and has a concave interior wall. As illustrated in FIG. 10, the geometry of each flared section 226a, 226b, 226c, 226d depends on the shape of each respective mound-shaped portion 220a, 220b, 220c, 220d.

The tip receiving section 228 extends from the flared section 226 to an end wall. The tip receiving section 228 has a cylindrical shape with a radius r_{c}. The tip receiving section 228 is sized and shaped to receive the tip of the guidewire. In one embodiment, the radius r_{c} of the tip receiving section 228 may vary along the length of the tip receiving section 228.

In-use, the first wire-shaping portion 218a having the first set of mound-shaped portions 220 and the first aperture 224 defined therein allows shaping a guidewire in function of the shape of one of the first set of mound-shaped portions 220 by using a single first aperture 224. The guidewire may be inserted into the first aperture 224 such that the distal end of the guidewire is placed into the tip receiving section 228. The guidewire may be curved by applying a force on the working section of the guidewire towards the working face 212 while another portion of the working section of the guidewire abuts on and follows the shape of a desired flared section 226a, 226b, 226c, 226d of the respective mound-shaped portions 220a, 220b, 220c and 220d.

The second wire-shaping portion 218b comprises a second mound-shaped portion 230 and has a second aperture 234 defined therein.

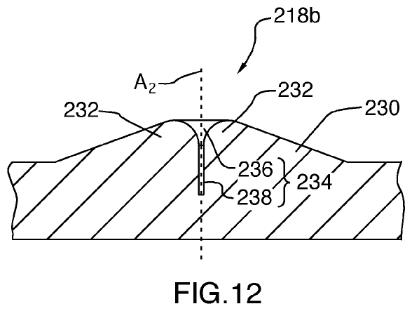

The second mound-shaped portion 230 projects from the working face 212 of the body 202 and away from the bottom face 214 along a second axis A₂ (as depicted in FIG. 12). The second-mound shaped portion 230 is symmetrical around the second axis A₂. The second mound-shaped portion 230 is shaped as a conical frustum i.e., a truncated cone, and has a top portion 232.

The top portion 232 is smooth and generally has an arcuate shape. The shape of the top portion 232 allows giving a smooth shape to a portion of the working section of the guidewire that abuts on the top portion 232 when a force is exerted on the guidewire towards the working face 212 when the guidewire is inserted into the second aperture 234.

The second aperture 234 extends from the second mound-shaped portion 230 through the working face 212 towards the transverse face 214.

The second aperture 234 is funnel shaped. The second aperture 234 comprises a flared or conical frustum shaped section 236 extending from the second mound-shaped portion 230, and a tip receiving or tubular section 238 extending from the flared section 236.

The flared section 236 of the second aperture 234 (as best seen in FIG. 12) extends from top portion 232 of the second mound-shaped portion 230 towards the bottom face 214.

The tip receiving section 238 extends from the flared section 236 to an end wall (not numbered). The tip receiving section 238 has a cylindrical shape or tubular shape and is sized to receive a tip of a guidewire.

It should be noted that the length of the tip receiving section 238 is greater than the length of the tip receiving section 228 due to the second mound-shaped portion 230, which allows the second aperture 234 to receive a greater length of guidewire compared to the first aperture 224. In another embodiment, the length of the tip receiving section 228 may be equal to the length of the tip receiving section 238 or greater than the length of the tip receiving section 238.

While the second aperture 234 is depicted as being defined at the center of the second mound-shaped portion 230, it is contemplated that the second aperture 234 may be defined offset from the center of the second mound-shaped portion 230.

The third wire-shaping portion 218c has a third aperture 244 defined therein. The third-wire shaping portion 218c does not have a mound-shaped portion.

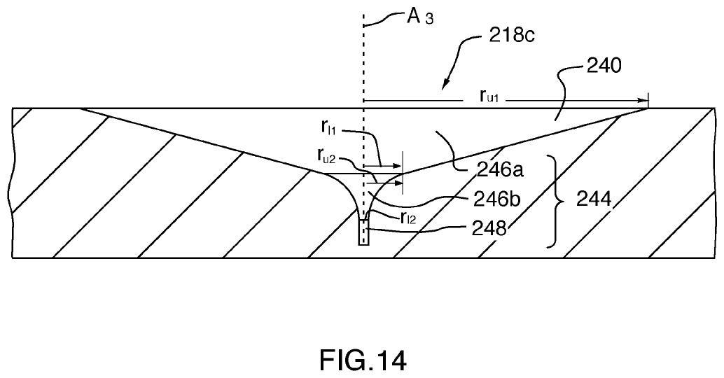

The third aperture 244 extends from the working face 212 of the body 202 towards the transverse face 214 along the third axis A₃ (as depicted in FIG. 14).

The third aperture 244 has three sections: a first flared section 246a, a second flared section 246b and a tip receiving section 248.

The first flared section 246a is shaped as an inverted conical frustum, i.e. a truncated cone, having an upperbase radius rᵤ₁ at the working face 212 and a lower base radius rₗ₁ below the working face 212 towards the bottom face 214. While the first flared section 246a is depicted as extending symmetrically from the working face 212, i.e. the upper base radius rᵤ₁ is constant in every direction, it is contemplated that the first flared section 246a may not extend symmetrically, i.e. the upper base radius rᵤ₁ may vary depending on the radial direction.

The second flared section 246b is shaped as an inverted conical frustum, i.e. an inverted truncated cone having an upper base radius rᵤ₂ at the connection with the lower base radius rₗ₁ of the first flared section 246a and a lower base radius rₗ₂ at the connection with the tip receiving section 248. The upper base radius rᵤ₂ of the second flared section 248b corresponds to the lower base radius rₗ₁ of the first flared section 246a.

In one embodiment, the first flared section 246a and the second flared section 246b are a single flared section.

The tip receiving section 248 extends from the second flared section 246b to an end wall (not numbered). The tip receiving section 248 is similar to the tip receiving section 238, i.e. the tip receiving section 248 has a cylindrical shape and is sized to receive a tip of a guidewire. It is contemplated that other shape of the tip receiving section 248 are possible.

The fourth wire-shaping portion 218d comprises a helicoid-shaped portion 250 and a fourth aperture 254 defined therein.

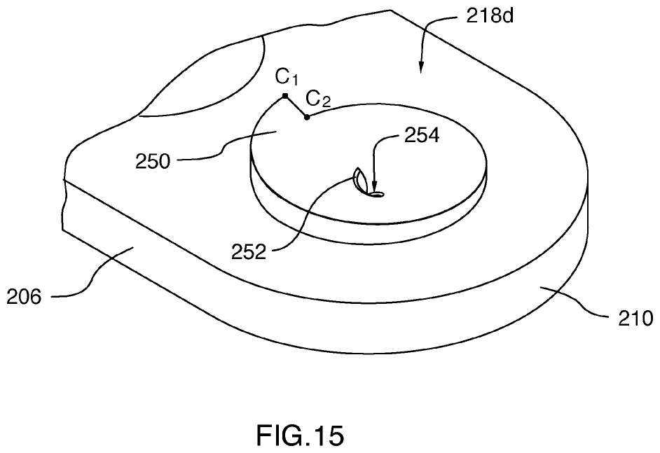

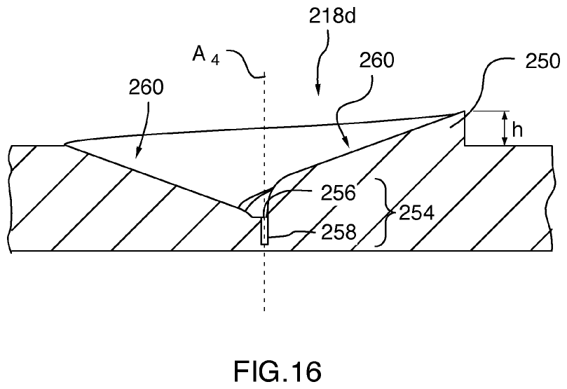

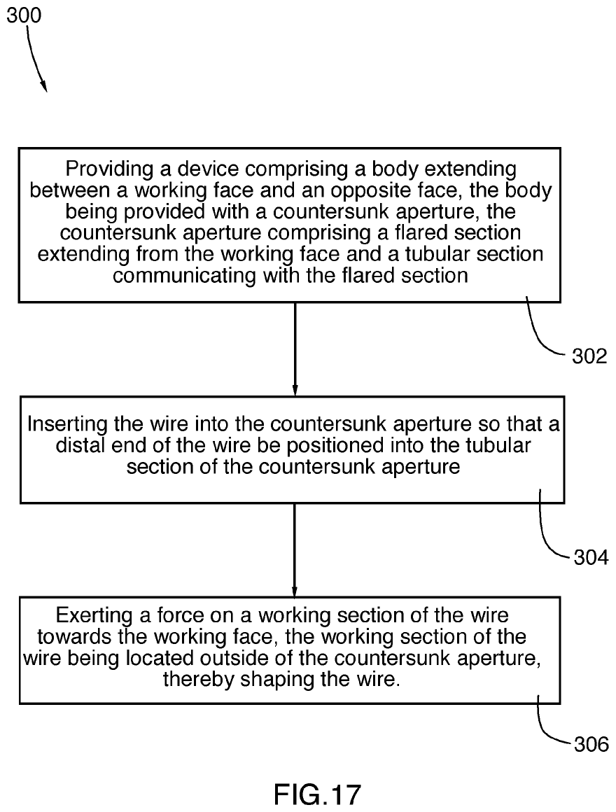

The helicoid-shaped portion 250 projects from the working face 212 of the body 202 and away from the bottom face 214 along a fourth axis A₄ (only depicted in FIG. 16). As best seen in FIG. 16 and FIG. 17, the helicoid-shaped portion 250 has a helicoid-shaped surface, wherein the height h of the helicoid-shaped portion 250 at a point C₁ at a circumference decreases gradually along the circumference of the helicoid-shaped portion 250 until being flush with the working face 212. In the embodiment illustrated in FIG. 15, the height h of the helicoid-shaped portion 250 is at its maximum at point C₁ and continuously decreases along an anti-clockwise circumference until reaching point C₂ which is flush with the working face 212 and radially aligned with point C₁, i.e. the height h at point C₂ is equal to zero.

The fourth wire-shaping portion 218d has a fourth aperture 254 defined therein. The fourth aperture 254 extends from the helicoid-shaped portion 250 to the working face 212 towards the bottom face 214 along the fourth axis A₄.

The fourth aperture 254 has a flared section 256 and a tip receiving section 258.

The flared section 256 follows the shape of the helicoid-shaped portion 250 such that the geometry of an interior wall 260 of the fourth aperture 254 varies with the circumference of the helicoid-shaped portion 250. The tip receiving section 258 extends from a portion of the flared section 256 to an end wall (not depicted).

The tip receiving section 258 of the fourth aperture 254 is similar to the tip receiving section 248, i.e. the tip receiving section 258 has a cylindrical shape sized and shaped to receive a tip of a guidewire.

In-use, the gradually decreasing height of the helicoid-shaped portion 250 allows to dictate the geometrical characteristics of a guidewire that is inserted into the fourth aperture 254 and being been shaped, bent or curved using the device 200. Thus, the guidewire may be given a different shape depending the direction where a force is exerted on the working section of the guidewire pressed against the wall 260.

In one embodiment, the helicoid-shaped portion 250 is provided with a plurality of notches in the surface each radially extending along the helicoid-shaped portion 250 from the edge of the helicoid-shaped portion 250 down to the tip receiving section 258, at a respective radial position about the circumference of the helicoid-shaped portion 250. The shape and size of each notch is chosen so as to receive at least a portion of a guidewire to be shaped therein. For example, a notches may be provided with a half cylindrical shape for receiving a cylindrical or tubular guidewire therein. Each notch allows for preventing a guidewire to slip while being shaped and the radial position of each notch defines a predetermined shape for the guidewire.

While the device 200 has been described with four wire-shaping portions 218a, 218b, 218c and 218d, it should be noted that the device 200 may have at least one of the four-wire shaping portions 218a, 218b, 218c and 218d.

FIG. 17 illustrates an embodiment of a method 300 for shaping, bending and/or curving a guidewire in accordance with one non-limiting embodiment.

At step 302, a device for shaping a wire such as the device 100, the device 150 or the device 200 is provided. The wire may be a guidewire. As described above, the guidewire shaping device may comprise a single wire-shaping portion having a single aperture defined therein. Alternatively, the guidewire shaping device may comprise a plurality of wire-shaping portions, each having one aperture.

At step 304, the guidewire to be shaped is inserted into an aperture so that the distal end of the guidewire be positioned into the tip receiving section of the aperture.

In one embodiment, the tubular section of the aperture is terminated by an end wall. In this case, the step 304 comprises the insertion of the distal end of the guidewire into the tubular section of the aperture. In one embodiment, the guidewire is inserted into the aperture so that the distal end of the guidewire does not abut against the end wall. In another embodiment, the guidewire is inserted into the aperture so that its distal end abuts against the end wall.

In another embodiment in which the aperture extends through the body, the guidewire may be inserted into the aperture so that the distal end of the guidewire be positioned outside of the body while a portion of the guidewire be positioned within the tubular section of the aperture. Alternatively, the guidewire may be inserted into the aperture so that the distal end of the guidewire be positioned within the tubular section of the aperture.

A force is applied on a working section of the guidewire at step 306 in order to shape, curve and/or bend a section of the guidewire such as the distal end of the guidewire has the shape of the tubular section. The working section of the guidewire corresponds to the section of the guidewire located outside of the aperture of the body once the distal end of the guidewire has been introduced into the tubular section of the aperture. In one embodiment, the working section of the guidewire corresponds to the section of the guidewire located outside of the body and adjacent to the working face of the body.

The force applied on the working section of the guidewire is exerted in direction of the working face of the body. As a result of the force exerted on the working section of the guidewire, the guidewire located into the tubular section of the aperture abuts against the wall surrounding the tubular section, thereby maintaining in position the distal end of the guidewire into the tubular section of the aperture. In an embodiment in which the aperture does not extend through the whole thickness of the body, the lateral wall of the distal end of the guidewire abuts against the wall surrounding the tubular section of the aperture. In an embodiment in which the aperture extends through the whole thickness of the body and the distal end of the guidewire is positioned outside of the body, the lateral wall of the guidewire abuts against the wall surrounding the tubular section of the aperture.

In one embodiment, the aperture extends from a mound-shaped portion, and a force may be applied to a section of the guidewire towards the mound-shaped portion such that the working section of the guidewire takes the shape of the mound-shaped portion, where the mound-shaped portion has an arcuate or smooth top portion. In one embodiment, the mound-shaped portion could be shaped as a conical frustum, or could have an helicoid-shaped surface.

By increasing the force exerted on the working section of the guidewire, the guidewire is curved. In one embodiment, the force is exerted on the working section of the guidewire until at least one point of the working section of the guidewire be in physical contact with the working face of the body.

In one embodiment, the force for shaping, bending or curving the guidewire is applied by the finger of a user on the working section of the guidewire. In this case, the user may press the guidewire with a finger until his/her finger and/or the guidewire contacts the working face of the body. In another embodiment, the force for shaping, bending or curving the guidewire may be applied via a pressure tool manipulated by a user. As a non-limiting example, a slotted and hinged clamp may be used, where the user may apply a force by pinching the clamp while the guidewire is position into a slot of the clamp.

In one embodiment, the method 300 may be used for shaping a guidewire such as a guidewire for guiding a catheter.

In one embodiment, the device 100, the device 150 and the device 200 are combined with the method 300 for shaping, bending, or curving a guidewire such as a guidewire for guiding a catheter used in cardiovascular, urological, gastrointestinal, neurovascular, and ophthalmic application.

In one embodiment, the device 102 and the method 300 could be used for shaping, bending, or curving a mechanical waveguide adapted to propagate mechanical waves such as shockwaves used for the treatment of chronic total occlusions.

The embodiments of the technology described above are intended to be exemplary only. The scope of the present technology is therefore intended to be limited solely by the scope of the appended claims.

I/We claim:

1. A device for shaping a wire comprising:
a body having a working face;
the body comprising a wire-shaping portion, the wire-shaping portion having an aperture defined therein, the aperture extending from the working face along an aperture axis, the aperture comprising:
a flared section; and
a tip receiving section connected to the flared section, the tip receiving section being shaped and sized for receiving a tip of the wire therein and maintaining a position of the tip of the wire while a bending force is exerted on the wire.

2. The device of claim 1, wherein
the wire-shaping portion further comprises a mound-shaped portion projecting from the working face; and wherein
the aperture extends from the mound-shaped portion.

3. The device of claim 2, wherein the mound-shaped portion has a conical frustum shape, the conical frustum having: a lower base and an upper base, the lower base being located below the upper base.

4. The device of claim 2, wherein the mound-shaped portion has a pyramidal-frustum shape.

5. The device of any of claims 2 to 4, wherein the mound-shaped portion has an arcuate top portion.

6. The device of claim 2, wherein the mound-shaped portion has a helicoid-shaped surface.

7. The device of any of claims 2 to 5, wherein a height of the mound-shaped portion varies along a circumference thereof.

8. The device of any of claims 2 to 5, wherein the mound-shaped portion is a plurality of mound-shaped portions.

9. The device of any of claims 2 to 8, wherein the flared section has a conical frustum shape, the conical frustum having: a lower base, and an upper base, the upper base being located below the lower base.

10. The device of any of claims 1 to 9, wherein the axis of the aperture is orthogonal to the working face.

11. The device of claim 8, wherein the flared section is a plurality of flared sections each extending from a respective one of the plurality of mound-shaped portions.

12. The device of any of claims 1 to 9, wherein the aperture extends along only a section of a thickness of the body, the tip receiving section extending up to an abutment wall for abutting the tip of the wire thereon.

13. The device of claim 12, wherein the abutment wall is flat.

14. The device of any one of claims 1 to 13, wherein the tip receiving portion has a cylindrical shape.

15. The device of any one of claims 7 to 14, further comprising a plurality of visual indicators each for identifying respective dimensions of the aperture and the tip-receiving section.

17. The device of any one of claims 1 to 15, wherein the wire is a guidewire for guiding a catheter.

18. The device of any one of claims 1 to 17, wherein the body is made at least partially of one of: a medical-grade material, a biocompatible material and a sterilizable material.

19. The device of claim 18, wherein the material is polyether ether ketone.

20. A method for shaping a wire, the method comprising:
providing a device comprising a body having a working face and a wire-shaping portion, the wire-shaping portion having an aperture defined therein, the aperture extending from the working face towards the bottom face along an axis, the aperture comprising: a flared section, and a tip receiving section connected to the flared section, the tip receiving section being shaped and sized for receiving a tip of the wire therein and maintaining a position of the tip of the wire while a bending force is exerted on the wire;
inserting the wire into the aperture such that a portion of the wire is positioned within the tip receiving section of the aperture; and
exerting a force on a working section of the wire towards the working face, the working section of the wire being located outside of the aperture, thereby shaping the wire.

21. The method of claim 20, wherein said inserting the wire into the aperture comprises positioning a distal end of the wire into the tip receiving section of the aperture.

22. The method of claim 21, wherein said inserting the wire into the aperture comprises abutting the distal end of the wire against an end wall of the tip receiving section of the aperture.

23. The method of claim 20, wherein said inserting the wire into the aperture comprises inserting the distal end of the wire into the tip receiving section until the distal end extends outside of the body.

24. The method of any one of claims 21 to 23, wherein the exerting the force comprises abutting at least a portion of the working section of the wire against the working face of the body.

25. The method of any one of claims 21 to 24, wherein the wire-shaping portion further comprises a mound-shaped portion; and wherein
the exerting the force towards the working face comprises abutting another portion of the working section of the wire on the mound-shaped portion.

26. The method of any one of claims 21 to 25, wherein the wire is a guidewire for guiding a catheter.
